(19) United States
(12) Patent Application Publication
(10) Pub. No.: US 2020/0003755 A1
(43) Pub. Date: Jan. 2, 2020

(54) NON-INVASIVE AND MINIMALLY-INVASIVE DETECTION OF SERUM IRON IN REAL TIME

(71) Applicants: Ghassan S. Kassab, La Jolla, CA (US); Ali Dabiri, San Diego, CA (US)

(72) Inventors: Ghassan S. Kassab, La Jolla, CA (US); Ali Dabiri, San Diego, CA (US)

(21) Appl. No.: 16/502,005

(22) Filed: Jul. 2, 2019

Related U.S. Application Data
(60) Provisional application No. 62/799,159, filed on Jan. 31, 2019.
(60) Provisional application No. 62/701,073, filed on Jul. 20, 2018.
(60) Provisional application No. 62/693,367, filed on Jul. 2, 2018.

(51) Int. Cl. G01N 33/49; G01N 21/64; G01N 21/3581; G01N 21/31

(52) U.S. Cl. CPC G01N 33/4925; G01N 21/6404; G01N 21/3103; G01N 21/3581; G01N 21/64

(57) ABSTRACT

Non-invasive and minimally-invasive detection of serum iron in real time. In a method for detecting serum iron content disclosed herein, the method includes positioning a device relative to a nonpigmented epithelial layer covering capillaries of a mammalian subject, operating the device to obtain optical data relating to the capillaries, and determining serum iron content of blood within the capillaries based upon the optical data.

### PRIORITY

The present application is related to, and claims the priority benefit of, U.S. Provisional Patent Application Ser. No. 62/799,159, filed Jan. 31, 2019, U.S. Provisional Patent Application Ser. No. 62/701,073, filed Jul. 20, 2018, and U.S. Provisional Patent Application Ser. No. 62/693,367, filed Jul. 2, 2018. The contents of each of these applications are incorporated into the present disclosure by reference in their entirety.

### BACKGROUND

Nearly two billion people and approximately 300 million children globally are afflicted with iron deficiency. Lack of iron causes anemia, impairs cognitive and behavioral development in childhood, compromises immune responsiveness, diminishes physical performance, and, when severe, increases mortality among infants, children, and pregnant women. Most of those affected are unaware of their lack of iron, in part because detection of iron deficiency requires a blood test. It is becoming increasingly important to screen these individuals to reduce medical cost and avoid chronic disease conditions. There are limited settings of laboratory infrastructure for standard blood-based tests around the World to accomplish this important screening test. Non-invasive screening is likely to be more acceptable to children and many other populations than methods requiring finger or vein puncture.

Presently, there are commercially available iron assay kits in the market. The disadvantage is the need for a blood sample and the time of the assay, which takes a minimum of one hour to perform. The kit measures iron in the linear range of 0.4 to 20 nmol in 50 μl sample. The assay produces a stable colored complex at 593 nm wavelength that can be detected with a photo detector.

In view of the same, there is a need for non-invasive and minimally invasive methods to provide a rapid, easy to use means for point-of-care (POC) screening for iron deficiency in resource-limited settings lacking laboratory infrastructure.

### BRIEF SUMMARY

The present disclosure includes disclosure of two different mechanisms to detect serum iron content in real time, namely non-invasive mechanisms/methods and minimally-invasive mechanisms/methods.

The present disclosure includes disclosure of a method for detecting serum iron content, comprising positioning a device relative to a nonpigmented epithelial layer covering capillaries of a mammalian subject, operating the device to obtain optical data relating to the capillaries, and determining serum iron content of blood within the capillaries based upon the optical data.

The present disclosure includes disclosure of a method further comprising the step of determining whether or not the mammalian subject is anemic based upon the determined serum iron content.

The present disclosure includes disclosure of a method, wherein the step of operating the device comprises operating a fluorescence spectroscopy device.

The present disclosure includes disclosure of a method, wherein the step of operating the device comprises operating the device to illuminate and acquire a fluorescence emission spectra from the subject.

The present disclosure includes disclosure of a method, wherein the step of operating the device comprises operating an optical fiber probe of the device to illuminate and acquire the fluorescence emission spectra from the subject.

The present disclosure includes disclosure of a method, wherein the step of operating the device comprises operating the device to obtain the optical data relating to the presence of zinc protoporphyrin of the blood.

The present disclosure includes disclosure of a method, wherein the step of positioning is performed by positioning the device relative to a lower lip of the mammalian subject.

The present disclosure includes disclosure of a method, wherein the step of operating the device comprises operating a terahertz spectroscopy device.

The present disclosure includes disclosure of a method, wherein the step of operating the device comprises operating the device to illuminate and acquire a terahertz emission spectra from the subject.

The present disclosure includes disclosure of a method, wherein the step of operating the device comprises operating an optical fiber probe of the device to illuminate and acquire the terahertz emission spectra from the subject.

The present disclosure includes disclosure of a method, wherein the step of operating the device comprises operating the device to obtain the optical data relating to an intensity of the terahertz emission spectra, whereby the intensity corresponds to a concentration of the serum iron content of the blood.

The present disclosure includes disclosure of a method for detecting serum iron content, comprising obtaining blood from a mammalian subject, operating a device to excite electrons within the blood and to measure a wavelength of emitted energy during a return of the excited electrons to a ground state, and determining serum iron content of the blood based upon wavelength of the emitted energy.

The present disclosure includes disclosure of a method, wherein the step of operating the device comprises operating an inductively coupled plasma atomic emission spectroscopy (ICP-AES) device.

The present disclosure includes disclosure of a method, wherein the step of operating the device comprises operating an inductively coupled plasma atomic optical spectroscopy (ICP-AOS) device.

The present disclosure includes disclosure of a method for detecting serum iron content, comprising obtaining blood from a mammalian subject, operating a device to obtain data relating to the blood, the data selected from the group consisting of viscosity data and conductance data, and determining serum iron content of the blood based upon the obtained data.

The present disclosure includes disclosure of a method, wherein the step of operating the device comprises operating a device configured to generate a magnetic field while obtaining the viscosity data.

The present disclosure includes disclosure of a method, wherein the step of operating the device comprises (a) obtaining first viscosity data relating to the blood using a device configured to obtain viscosity data, and (b) obtaining second viscosity data relating to the blood using the device configured to obtain viscosity data while a magnetic field is applied to the blood.

The present disclosure includes disclosure of a method, wherein the step of determining serum iron content is performed by comparing the first viscosity data to the second viscosity data.

The present disclosure includes disclosure of a method, wherein the step of operating the device comprises operating a device configured obtain the conductance data.

The present disclosure includes disclosure of a method, wherein the obtained data comprises the conductance data, whereby relatively low conductance data is indicative of low serum iron content.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure of two different mechanisms to detect serum iron content in real time, namely non-invasive mechanisms/methods and minimally-invasive mechanisms/methods.

Non-Invasive Methods

The use of a patient's hair or nails (fingernails or toenails) to estimate the serum iron content is referenced herein. The possibility of an optical signature of the serum iron can also be explored if the goal is a non-invasive and real time assay. The present disclosure considers two optical approaches, namely florescence spectroscopy and Tera-Hertz (THz) spectroscopy. In these methods, the serum iron needs to have signature with very high sensitivity and specificity and the response needs to be linear with the concentration of the serum iron.

Detection of Iron Content in Hair/Nail by Magnetometer

Human serum is recognized as the “gold standard” to determine iron and other mineral levels. It is important to note that in the most widely-used test of serum ferritin level, the body iron status may not be accurately reflected due to various conditions, including pregnancy, acute or chronic inflammatory disease, malignancy, infection, renal failure, or malabsorption syndrome. Hair can be an attractive alternative due to its simplicity as a sample (easy to obtain, without trauma and/or discomfort), storage, transport and handling.

The determination of hair iron concentration necessitates a strict sampling regime, however, which is not practical. Historically, there has been little data associated with the use of hair iron concentration to define body iron status. In 1956, Duffield et al. concluded that hair iron concentration may not provide sufficient information regarding total body iron. In 1971, Lovric et al. measured the iron content of various hair segments of children with iron deficiency and iron overload and concluded that there was no significant association between the groups with respect to hair iron concentration. In subsequent years, however, Bisse et al. concluded that hair iron concentration is useful in the evaluation of body iron status. Sahin et al. studied the possible association between blood parameters and hair iron concentration in patient groups with different body iron contents through chemical analysis. The study population comprised of 25 patients (mean of 33 years) with iron deficiency anemia and 20 patients (mean of 22 years) with transfusion-related anemia that showed a difference in body iron content. The 21 healthy control group was formed of age (mean of 28 years) and gender-matched subjects with no history of underlying disease. The results showed measured mean hair iron ⁵⁶Fe and ⁵⁷Fe concentrations of the iron deficiency group were 5.08 and 6.03 μg/g, respectively, and in the transfusion-related anemia group these values were 28.9 and 29.4 μg/g, respectively. In the control group, the mean hair iron ⁵⁶Fe and ⁵⁷Fe concentrations were measured as 12.0 and 17.6 μg/g, respectively. The highest hair iron concentration (89.4 μg/g) was observed in transfusion-related anemia patients, whereas the lowest hair iron concentration (0.77 μg/g) was determined in the iron deficiency anemia group. The differences between the three groups with respect to hair iron ⁵⁶Fe and ⁵⁷Fe concentrations were found to be statistically significant. In addition, a positive correlation was determined between hair iron ⁵⁶Fe and ⁵⁷Fe concentrations and serum iron, ferritin level, transferrin saturation, MCV and MCH values, which are the most important parameters showing body iron content. This study concluded that patient groups with different body iron content had a significant difference in hair iron concentration and these values were correlated with laboratory markers of body iron content. These results support the view that hair sampling can be used as a marker of body iron content.

In another study, Claudio et al. developed a method to determine iron in human hair samples by graphite furnace atomic absorption spectrometry (GF AAS). They measured iron levels in hair samples from 20 pre-adolescent, menstruating girls in schools in Brazil. The concentration range was 14-26 μg/g. Baranowska et al. analyzed hair samples collected from the inhabitants of Poland by x-ray fluorescence spectrometry and obtained an average concentration of 36.3 μg/g for Fe in hair samples.

Human nail (fingernails and toenails) can also be an attractive alternative due to its simplicity as a sample (easy to obtain, without trauma and/or discomfort), storage, transport and handling. Sobolewski et al. measured the iron content of healthy and iron deficient individual nails. The iron content of the nails ranged from 6 to 26 μg/g of nail for the women and 6 to 23 μg/g for the men in healthy individual group. This value dropped to less than 4 μg/g for the iron deficient subjects. In iron-depleted and iron-sufficient subjects there was a correspondence between iron content of the nails and bone marrow iron, serum iron and TIBC.

The major disadvantage of these methods is the need to transport the hair/nail samples to an analytical laboratory for testing which is time consuming, expensive and the facility may not be accessible in developing countries.

The present disclosure includes disclosure of a magnetometer to detect iron content in human hair/nail to screen for iron deficient patients. The device is a portable unit that can be operated with a trained technician.

One example of a magnetometer can be vibrating sample magnetometer (VSM). A VSM is a scientific instrument that measures magnetic properties is. Simon Foner at MIT Lincoln Laboratory invented VSM in 1955 and reported it in 1959. A sample is first magnetized in a uniform magnetic field. It is then sinusoidally vibrated, typically through the use of a voice coil actuator. The induced voltage in the pickup coil is proportional to the sample's magnetic moment, but does not depend on the strength of the applied magnetic field. In a typical setup, the induced voltage is measured with a lock-in amplifier using the vibration frequency as the reference.

Florescence Spectroscopy

In the developing red blood cell, the insertion of iron into protoporphyrin IX is the final step in the production of haem for incorporation into haemoglobin. If iron is unavailable, divalent zinc is incorporated instead, producing zinc protoporphyrin, which persists for the life of the red blood cell as a biochemical indicator of functional iron deficiency. In regions with endemic for malaria and other infections, the World Health Organization recommends measurement of the red blood cell zinc protoporphyrin as the preferred indicator to screen children for iron deficiency. In the United States, the American Academy of Pediatrics recommends universal screening for iron deficiency at one year of age, and the use of red blood cell zinc protoporphyrin for this purpose has been suggested. Screening for iron deficiency using red blood cell zinc protoporphyrin has recently been proposed as standards. With blue light excitation, zinc protoporphyrin fluoresces, while haem does not. The feasibility to detect this fluorescence is included in the present disclosure, where an optical fiber probe can be used to illuminate and acquire the fluorescence emission spectra from the lower lip, where only a thin, nonpigmented epithelial layer covers the blood-filled capillaries perfusing the underlying tissue. A portable fluorescence spectroscopy device would be ideal for use in regions where medical facilities are not readily available or accessible.

Terahertz (THz) Spectroscopy

THz spectroscopy and imaging (imaging at frequencies around 10¹² Hz) is a novel technique for medical imaging. It uses non-ionizing radiation and can safely be used for imaging different types of tissue, such as normal cells and tumors; the contrast between tissue types is thought to occur due to differences in water content, protein density or cellular structure. Penetration of tissue depends on the fat and water content and can reach a depth ranging from several hundred microns to several millimeters.

Terahertz spectroscopy has been used to characterize the blood. The complex optical constants of blood and its constituents, such as water, plasma, and red blood cells (RBCs), were obtained in the THz frequency region. The volume percentage of RBCs in blood was extracted and compared with the conventional RBC counter results. The THz absorption constants are shown to vary linearly with the RBC concentration in both normal saline and whole blood. The feasibility of this technique is referenced herein to detect the iron deficiency and its sensitivity and specificity. An optical fiber probe is used to illuminate and acquire the terahertz emission spectra from the lower lip, where only a thin, nonpigmented epithelial layer covers the blood-filled capillaries perfusing the underlying tissue. The rationale is that the optical signature intensity is proportional to the concentration of the RBC iron concentration. A portable THz spectroscopy device would be ideal for use in regions where medical facilities are not readily available or accessible.

Minimally-Invasive Methods

Small blood samples are necessary for in vitro analysis, as referenced herein. Three methods, namely Inductively Coupled Plasma Atomic Emission (or Optical) Spectroscopy (ICP-AES, or ICP-AOS), serum viscosity change in a magnetic field, and bio-impedance are disclosed herein.

Traditionally, serum would need to be separated from the blood in order to measure the iron level in blood due to transferrin, which is one of three markers doctors usually order to find the status of the iron in the body (the other two are TIBC and ferritin). In other situations, such as regions with endemics for malaria and other infections, the World Health Organization (WHO) recommends measurement of the red blood cell zinc protoporphyrin as the preferred indicator to screen children for iron deficiency.

In the methods noted below, blood samples can be used directly rather than serum.

ICP-AES/ICP-AOS

ICP-AES/ICP-AOS are emission spectrophotometric techniques, exploiting the fact that excited electrons emit energy at a given wavelength as they return to a ground state after excitation by high temperature argon plasma. The rationale of this process is that each element emits energy at specific wavelengths peculiar to its atomic character. The energy transfer for electrons when they fall back to the ground state is unique to each element as it depends upon the electronic configuration of the orbital. This technique has been used to analyze biological samples. The analysis can be made in real time with high detection sensitivity. The unit size is tabletop, although some portable systems have been built for metallic element analysis in the warehouses. This technique can be utilized to detect serum iron and its sensitivity with different blood samples. Once satisfied, the unit can be tailored for this purpose and make it smaller for the bed-side application.

Assays in Magnetic Fields

Physicists Rongjia Tao and Ke Huang took donated blood and then measured its viscosity in a small tube used for that purpose. They then applied a 1.3 Tesla magnetic field to the tube (this is about the strength of the magnetic field used in a typical MRI scanner), with the field aligned with the direction of blood flow, for one minute and found that the viscosity decreased by 20-30%. This effect lasted for about 2 hours. The rationale comes from the blood cells clumping together, mostly in a line, like box cars on a train. The cells moving together as a train produces less resistance than if they were all bouncing around separately. Further, they tend to flow more down the middle of the tube, reducing friction with the tube wall. The glass tube used in the study was larger than the smallest arteries in humans. It is postulated that the viscosity in this set-up is directly proportional to the iron content of the RBC in the blood. This method can be used to determine the iron deficiency of the blood. This concept, as noted in the present disclosure, can be used to measure the iron content in the serum in a magnetic field if the interest is the measurement if the iron in the serum. The change of viscosity can be measured by a viscometer. The magnet with the 1.3 T strength can be rather small since the core of the magnet where the sample is placed can be as small as 0.5 cm in diameter. The best candidate is neodymium magnets.

Neodymium magnets, invented in the 1980s, are the strongest and most affordable type of rare-earth magnet. They are made of an alloy of neodymium, iron, and boron (Nd₂Fe₁₄B), sometimes abbreviated as NIB. Neodymium magnets are used in numerous applications requiring strong, compact permanent magnets, such as electric motors for cordless tools, and hard disk drives. They have the highest magnetic field strength and have a higher coercivity (which makes them magnetically stable). Since their prices became competitive in the 1990s, neodymium magnets have been replacing ferrite magnets in the many applications in modern technology requiring powerful magnets. Their greater strength allows smaller and lighter magnets to be used for a given application. The speakers use this kind of magnets with about 1.4 T magnetic strength and the sizes are not big by any standard.

Bio-Impedance

A bio-impedance method can also be used to detect iron levels in real time. Iron is electrically conductive, and the concentration of iron is proportional to electrical conductance (inverse of impedance); i.e., less iron implies lower electrical conductance. As such, operating a conductance device on a blood sample can result in obtaining conductance data, and relatively low conductance data is indicative of low iron concentration.

While various embodiments of methods and devices for the non-invasive detection of serum iron in real time have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

### REFERENCES

1. Sahin C. et al., Measurement of hair iron concentration as a marker of body iron content, Biomedical reports, Volume 3, Issue 3, DOI: 10.3892/br.2015.419, 2015.
2. Duffield J and Green P T: The iron content of human hair. II. Individuals with disturbed iron metabolism. Can Serv Med J 12: 987-996, 1956.
3. Lovric V A and Pepper R: Iron content of hair in children in various states of iron balance. Pathology 3: 251-256, 1971.
4. Bisse E, Renner F, Sussmann S, Scholmerich J and Wieland H: Hair iron content: possible marker to complement monitoring therapy of iron deficiency in patients with chronic inflammatory bowel diseases, Clin Chem 42: 1270-1274, 1996.
5. Claudio L. Donnici et al. Fast Determination of Iron and Zinc in Hair and Human Serum Samples After Alkaline Solubilization by GF AAS, J. Braz. Chem. Soc., Vol. 27, No. 1, 119-126, 2016.
6. Baranowska, I.; Barchanski, L.; Bak, M.; Smolec, B.; Mzyk, Z.; Pol. J. Environ. Stud., 13, 369, 2004.
7. Sobolewski, S. et al., Human nails and body iron, j. Clinical Pathology, 31, 1068-1072, 1978.
8. Wikipedia, VSM, accessed in July 2019.

1. A method for detecting serum iron content, comprising:
positioning a device relative to a nonpigmented epithelial layer covering capillaries of a mammalian subject;
operating the device to obtain optical data relating to the capillaries; and
determining serum iron content of blood within the capillaries based upon the optical data.

2. The method of claim 1, further comprising the step of:
determining whether or not the mammalian subject is anemic based upon the determined serum iron content.

3. The method of claim 1, wherein the step of operating the device comprises operating a fluorescence spectroscopy device.

4. The method of claim 3, wherein the step of operating the device comprises operating the device to illuminate and acquire a fluorescence emission spectra from the subject.

5. The method of claim 4, wherein the step of operating the device comprises operating an optical fiber probe of the device to illuminate and acquire the fluorescence emission spectra from the subject.

6. The method of claim 4, wherein the step of operating the device comprises operating the device to obtain the optical data relating to the presence of zinc protoporphyrin of the blood.

7. The method of claim 1, wherein the step of positioning is performed by positioning the device relative to a lower lip of the mammalian subject.

8. The method of claim 1, wherein the step of operating the device comprises operating a terahertz spectroscopy device.

9. The method of claim 8, wherein the step of operating the device comprises operating the device to illuminate and acquire a terahertz emission spectra from the subject.

10. The method of claim 9, wherein the step of operating the device comprises operating an optical fiber probe of the device to illuminate and acquire the terahertz emission spectra from the subject.

11. The method of claim 9, wherein the step of operating the device comprises operating the device to obtain the optical data relating to an intensity of the terahertz emission spectra, whereby the intensity corresponds to a concentration of the serum iron content of the blood.

12. A method for detecting serum iron content, comprising:
obtaining blood from a mammalian subject;
operating a device to excite electrons within the blood and to measure a wavelength of emitted energy during a return of the excited electrons to a ground state; and
determining serum iron content of the blood based upon wavelength of the emitted energy.

13. The method of claim 12, wherein the step of operating the device comprises operating an inductively coupled plasma atomic emission spectroscopy (ICP-AES) device.

14. The method of claim 12, wherein the step of operating the device comprises operating an inductively coupled plasma atomic optical spectroscopy (ICP-AOS) device.

15. A method for detecting serum iron content, comprising:
obtaining blood from a mammalian subject;
operating a device to obtain data relating to the blood, the data selected from the group consisting of viscosity data and conductance data; and
determining serum iron content of the blood based upon the obtained data.

16. The method of claim 15, wherein the step of operating the device comprises operating a device configured to generate a magnetic field while obtaining the viscosity data.

17. The method of claim 15, wherein the step of operating the device comprises (a) obtaining first viscosity data relating to the blood using a device configured to obtain viscosity data, and (b) obtaining second viscosity data relating to the blood using the device configured to obtain viscosity data while a magnetic field is applied to the blood.

18. The method of claim 17, wherein the step of determining serum iron content is performed by comparing the first viscosity data to the second viscosity data.

19. The method of claim 15, wherein the step of operating the device comprises operating a device configured obtain the conductance data.

20. The method of claim 19, wherein the obtained data comprises the conductance data, whereby relatively low conductance data is indicative of low serum iron content.
